(19) United States
(12) Patent Application Publication
(10) Pub. No.: US 2002/0114832 A1
(43) Pub. Date: Aug. 22, 2002

(54) Pharmaceutical preparation comprising peppermint oil and caraway oil in delayed release form

(75) Inventors: Joachim Herrmann, St. Leon-Rot (DE); Rainer Oschmann, Landau (DE); Karl-Heinz Stumpf, Karlsruhe (DE)

Correspondence Address: Peter F. Corless, EDWARDS & ANGELL, LLP, Dike, Bronstein, Roberts & Cushman, IP Group, 130 Water Street, Boston, MA, 02109, US

(73) Assignee: Dr. Willmar Schwabe GmbH & Co.

(21) Appl. No.: 09/734,392

(22) Filed: Dec. 11, 2000

(51) Int. Cl. A61K 9/62; A61K 35/78

(52) U.S. Cl. 424/461; 424/747

(57) ABSTRACT

The invention provides improved pharmaceutical preparations comprising peppermint oil and caraway oil for the treatment of dyspeptic complaints and cramps in the gastrointestinal region. In particularly preferred embodiments, preparations of the invention comprise high dosages of peppermint oil and caraway oil in a delayed-release form which also preferably consists of an enteric-coated soft gelatin capsule. Related methods also are disclosed.

### BACKGROUND OF THE INVENTION

1. Field of the Invention

The invention relates to a pharmaceutical preparation comprising peppermint oil and caraway oil for the treatment of dyspeptic complaints and cramps in the gastrointestinal region. More specifically, in accordance with the invention, peppermint oil and caraway oil are highly-dosed and used in a delayed-release form in order to increase tolerance.

2. Background

Peppermint oil (Menthae piperitae aetheroleum) has been employed in various pharmaceutical preparations. It is obtained by means of distillation from the blooming tips of peppermint twigs and its essential components are alcohols such as menthol, esters such as menthylacetate, and ketones such as menthone.

It has been shown that peppermint oil is useful in the treatment of spasmodic complaints in the upper gastrointestinal tract (Federal Health Agency-Monography Bundesanzeiger No. 50 of Mar. 13, 1986), with reported effects which include among others: spasmolytic, carminative, and secretolytic. Further, peppermint oil has been employed in a form which is resistant to gastric juice, however, such use has only been associated with the indication colon irritable, i.e. intestinal disorders.

Caraway oil (Carvi aetheroleum) is obtained from ripe caraways and its main component is carvone. The fields of application for caraway oil are dyspeptic complaints, flatulence, sensation of repletion and spasmodic complaints in the gastrointestinal region (Federal Health Agency-Monography Bundesanzeiger No. 22 of February 1990).

The combination of peppermint oil and caraway oil also has been used in these fields of application (dyspeptic complaints, flatulence, sensation of repletion and spasmodic complaints in the gastrointestinal region). However, with a high dosage of these ethereal oils, notable side effects concerning the stomach have been reported. This also is reflected in a correction dated Sep. 1, 1990 of the above mentioned Federal Health Agency-Monography relating to peppermint oil (BAnz No. 164); in this correction the following is added: “Side-effects: with sensitive persons there is the possibility of stomach complaints.”

Thus, it would be highly desirable to develop improved preparations which comprise the naturally-occurring peppermint oil and caraway oil for use in the pharmaceutical industry which do not present undesirable side-effects. It also would be highly desirable to develop such new preparations in a delayed release form, which form would preferably have equivalent bioavailability to comparable preparations in an immediate release drug. It also would be particularly desirable to develop new coatings, e.g., enteric-coatings, for such preparations which do not require the use of organic solvents and/or organic materials. Such preparations would be highly useful as a pharmaceutical in the treatment of dyspeptic complaints and spasmodic complaints in the gastrointestinal region.

### SUMMARY OF THE INVENTION

We have now discovered an improved pharmaceutical preparation comprising peppermint oil and caraway oil for the treatment of dyspeptic complaints and cramps in the gastrointestinal region. Preparations of the invention do not cause undesirable side-effects in patients as have been observed using preparations of the prior art which employ peppermint oil and/or caraway oil as active ingredients. Additionally, preparations of the invention are formulated in delayed-release forms without impairing the bioavailability of the active ingredients. Preparations of the invention preferably employ both peppermint oil and caraway oil at relatively high dosage levels, yet surprisingly, without adversely affecting safety or tolerability.

Other aspects of the invention are discussed infra.

### DETAILED DESCRIPTION OF THE INVENTION

As noted above, the present invention provides an improved pharmaceutical preparation comprising peppermint oil and caraway oil for the treatment of dyspeptic complaints and cramps in the gastrointestinal region. Additionally, preparations of the invention are formulated in delayed-release forms without impairing the bioavailability of the active ingredients. As used herein, the term “delayed release” refers to enteric-coated capsules which are resistant to gastric juice, but which decompose readily in intestinal juice, e.g., within approximately 30 to 45 minutes. (See also, The U.S. Pharmacopoeia, for standard operating procedures, general drug release standards and related information relevant to delayed release enteric coated capsules.)

As noted above, preparations of the invention do not result in the undesired side-effects observed in preparations of the prior art which employ peppermint oil and/or caraway oil as active ingredients. For example, we carried out a study in 80 patients with enteric coated peppermint oil (90 mg) and caraway oil (50 mg). The results of the study showed that undesired side-effects were noted in only about 3% of the patient population. (See, e.g., Example 7b which follows.) In contrast, attention is drawn to Example 7(a), for its report of comparative data (capsules without a gastric resistant coating), according to which 8.5% of the patients noted undesired side effects.

Plant preparations which are standard in trade in the indication region “dyspepsia” consist of combinations of several plant active substances and usually contain tinctures of caraway seeds or peppermint leaves with a correspondingly low oil content or the ethereal oils thereof in dosages of about 1 to 10 mg per single dose. In contrast, preparations of the invention preferably employ both peppermint oil and caraway oil at relatively high dosage levels, yet surprisingly, without adversely affecting safety or tolerability.

Further, the present invention provides a gastrointestinal preparation which has only minimal side-effects (if any), is small and easy to take and which ensures an unchanged bioavailability of the active substances as compared to a comparable preparation in an immediate release dug. We have found that the active substances can be filled into soft gelatin capsules together with excipients and provided with several coatings which delay release.

Surprisingly, in spite of the coating, the ethereal oils in this high dosage preparation not only produce the expected effects in the intestines but also in the stomach. See, for instance, Example 1 below, where caraway oil and peppermint oil were administered in a high dosage preparation directly into the duodenum by means of probes. The results of that Example clearly demonstrate an alteration of the gastric motility pattern.

Another surprising fact was that soft gelatin capsules with 90 mg peppermint oil and 50 mg caraway oil which were coated with hydroxypropylmethylcellulosephthalate were bioequivalent to immediate release capsules with respect to the reabsorbed quantity (AUC). More specifically, there was a delay in the maximum plasma level but no loss of bioavailability (See, e.g., Example 6 below).

In particularly preferred embodiments of the present invention, the preparations comprise soft gelatin capsules which are provided with coatings which delay release and which therefore are well-tolerated by the stomach and which contain in addition to auxiliary agents the two active substances caraway oil and peppermint oil in a high dosage and which capsules can be administered for the treatment of gastrointestinal complaints.

In such preferred embodiments of the invention, peppermint oil is present in a dosage amount of between about 50 and about 180 mg per single dose, more preferably about 80 to about 150, most preferably about 90 mg; and caraway oil is present in a dosage amount of between about 25 and about 100 mg per single dose, more preferably about 45 to about 75, most preferably about 50 mg. Typically, excipients used are waxes and triglycerides as well as soybean oils, preferably medium-chained triglycerides, which are well known in the pharmaceutical and/or dietary supplement fields.

Preparations of the invention preferably further comprise a delayed-release coating. Generally preferred are those coatings which readily dissolve in the upper small intestine at a pH of about 5.0 to about 6.0. Particularly preferred coatings for use in preparations of the invention include cellulosephthalate derivatives such as celluloseacetatephthalate and hydroxypropylmethylcellulosesuccinate, as well as shellac. Preparations of the invention may optionally further comprise additives, e.g., antisticking agents such as talcum or plasticizers such as dibutylphthalate or diethylphthalate, triacetine or citric acid. Preferred amounts of such additives may be readily determined by the skilled artisan.

Shellac is a most particularly preferred coating for use in preparations of the invention. Formerly, shellac was a generally undesirable alternative for use as a coating for pharmaceutical capsules because, among other things, it was quite brittle. However, superior shellac coating compositions have been developed and are now commercially available from sources well known to the skilled artisan. Accordingly, preparations of the invention preferably comprise use of such a shellac coating composition.

In addition, another advantage of shellac is that shellac is a natural product. Though celluloseacetatephthalate, hydroxypropylmethylcellulosesuccinate or methylydroxypropylcellulosephthalate, provide suitable enteric coatings, these kinds of cellulose derivatives are partially synthetic products.

Yet another advantage of shellac is that shellac is considered a foodstuff additive in the United States. As such, there is no need for an FDA approval with respect to the use of shellac as a coating for a capsule such as a soft gelatin capsule in the field of dietary supplements.

In summary, shellac is a safe natural product which presents no side-effects when used as a coating for preparations of the invention.

Moreover, for use in particularly preferred embodiments of the invention, we have developed coatings which comprise aqueous shellac solutions or aqueous shellac dispersions. This provides yet another significant advantage in that there is no need for disposal of an organic solvent, thus reducing cost and environmental concerns. In addition, where an organic solvent is used, another notable disadvantage is that there are often traces of the various organic solvents remaining in the coating. In contrast, in preparations of the invention which comprise aqueous-based shellac coatings, the coating does not contain any residues of an organic solvent.

Further, performance of shellac-coated capsules can reasonably be equated to the in vivo data provided in the Examples below for the cellulose derivative coating. (See, e.g., Example 6.)

For instance, the peppermint oil/caraway oil preparations are in the form of soft gelatin capsules filled with a mixture of these two oils diluted only with medium chain triglycerides. The oil mixtures do not contain any further ingredients such as would be the case in tablets. In that the soft gelatin capsules coated with shellac have the same resistance in the artificial gastric juice and the same disintegration patterns in the artificial intestinal juice in comparison to capsules coated with a cellulose derivative, the analogy can be made that the in vivo data obtained for the capsules coated with the cellulose derivative are the same as for the capsules coated with shellac. Specifically, after dissolution of the capsule itself, the active ingredient is the combination of the peppermint oil and caraway oil. This combination corresponds to the composition of the peppermint oil and caraway oil in the case of a cellulose derivative being used as a coating. Thus, after dissolution of the coating, the capsules no longer differ from each other and one can reasonably anticipate that the in vivo activity data also will be similar.

The present invention also provides methods of treating dyspeptic complaints and spasmodic complaints in the gastrointestinal region. In preferred embodiments of the invention, such methods comprise administering a pharmaceutical preparation to a mammal, including a human, which preparation comprises therapeutic amounts of peppermint oil and caraway oil in a delayed release form in accordance with those preparations described above.

Any and all documents referenced herein shall be incorporated by reference. Additionally, the following non-limiting examples are illustrative of the invention.

### EXAMPLE 1

A mixture of 90 mg peppermint oil, 50 mg caraway oil and medium-chained triglycerides was administered directly into the duodenum by means of a probe introduced through the nose.

The stomach motility was determined by means of a manometry probe. The number of contractions, the sum and the mean values of the amplitudes as well as the motility index (=logarithm of: sum of the amplitudes×number of amplitudes+1) were evaluated.

Table 1 (number of contractions in the antrum), Table 2 (mean values of the amplitudes in the antrum) and Table 3 (motility index in the antrum) show that the intraduodenal administration of a peppermint oil and caraway oil mixture has a significant influence on gastric motility.

TABLE 1
Number of contractions in the antrum before and after intraduodenal
administration of the peppermint oil/caraway oil/triglyceride mixture.

| Period of Time (min) | Before Instillation | After Instillation | |
|---|---|---|---|
| 0-30 | 10.8 ± 4.3 | 10.8 ± 4.3 | n.s. |
| 30-60 | 12.8 ± 4.8 | 2.8 ± 1.7 | p < 0.05 |
| 60-90 | 14.5 ± 7.3 | 11.8 ± 7.5 | p < 0.005 |
| 90-120 | 47.1 ± 8.0 | 11.8 ± 4.4 | p < 0.005 |
| 120-150 | 55.6 ± 10.9 | 14.0 ± 7.1 | p < 0.002 |
| 0-150 | 137.6 ± 22.4 | 47.3 ± 19.2 | p < 0.002 |

TABLE 2
Mean values of the amplitudes in the antrum before and after
intraduodenal administration of the peppermint oil/caraway
oil/triglyceride mixture.

| Period of Time (min) | Before Instillation | After Instillation | |
|---|---|---|---|
| 0-30 | 23.4 ± 7.9 | 38.5 ± 7.9 | p < 0.005 |
| 30-60 | 45.0 ± 14.5 | 14.0 ± 10.1 | p < 0.05 |
| 60-90 | 26.2 ± 5.4 | 25.7 ± 11.0 | p < 0.005 |
| 90-120 | 43.6 ± 10.2 | 38.4 ± 8.0 | p < 0.002 |
| 120-150 | 54.9 ± 7.4 | 50.3 ± 10.8 | p < 0.002 |
| 0-150 | 58.3 ± 14.4 | 41.1 ± 6.1 | p < 0.002 |

TABLE 3
Motility indices in the antrum before and after intraduodenal
administration of the peppermint oil/caraway oil/triglyceride
mixture.

| Period of Time (min) | Before Instillation | After Instillation | |
|---|---|---|---|
| 0-30 | 5.85 ± 1.91 | 7.24 ± 1.04 | p < 0.01 |
| 30-60 | 7.33 ± 1.53 | 2.71 ± 1.71 | p < 0.05 |
| 60-90 | 6.91 ± 1.58 | 6.44 ± 1.47 | p < 0.005 |
| 90-120 | 11.17 ± 0.49 | 7.10 ± 1.59 | p < 0.002 |
| 120-150 | 11.73 ± 0.57 | 7.38 ± 1.66 | p < 0.002 |
| 0-150 | 12.68 ± 0.83 | 10.80 ± 0.90 | p < 0.002 |

### EXAMPLE 2

Gastric-juice resistant combination preparation made of peppermint oil and caraway oil.

| Composition: | | |
|---|---|---|
| Caraway oil | 50.0 | mg |
| Peppermint oil | 90.0 | g |
| Medium-chained triglycerides | 50.0 | g |

The mixture is filled into soft gelatin capsules which are coated with 18 mg hydroxypropylmethylcellulosephthalate and 3.6 mg dibutylphthalate. The capsules are stable for two hours in artificial gastric juice (0.1 N HCl) and decompose within 30 minutes in artificial intestinal juice (phosphate buffer pH 6.8).

### EXAMPLE 3

Coated soft gelatin capsules with shellac.

| Composition: | | |
|---|---|---|
| Caraway oil | 50.0 | mg |
| Peppermint oil | 90.0 | g |
| Medium-chained triglycerides | 50.0 | g |

The mixture is filled into soft gelatin capsules which are coated with a shellac solution, consisting of shellac (15 mg/capsule) and triethylcitrate dissolved in ethanol.

### EXAMPLE 4

Surprisingly, capsules containing a peppermint oil/caraway oil mixture can be coated in a simple way by use of an aqueous shellac solution or dispersion without attracting the gelatin capsules, i.e., it is not necessary to use ethanol solutions for the shellac coating.

| Composition: | | |
|---|---|---|
| 26% aqueous shellac solution | 230 | g |
| triethylcitrate | 3 | g |
| purified water | 227 | g |

To the shellac solution triethylcitrate is added as a plasticizer and is diluted with purified water. At an exhaust air temperature of 36±2° C., the solution is sprayed onto the capsules in a drum coater. At an amount of 12 mg coating/cm² on the capsule surface, the capsules are resistant to gastric juice (2 hours stable in the artificial gastric juice) and decompose in the artificial intestinal juice within 30 minutes.

### EXAMPLE 5

| Composition: | | |
|---|---|---|
| 20% aqueous shellac dispersion | 280 | g |
| triethylcitrate | 2.8 | g |
| talcum | 20 | g |
| purified water | 128.0 | g |

To the shellac dispersion triethylcitrate is added as a plasticizer and is diluted with purified water. Talcum is dispersed in order to avoid stickiness. The coating is carried out as described in Example 4 above.

### EXAMPLE 6

Bioavailability of delayed released peppermint oil/caraway oil soft gelatin capsules.

Enteric coating of peppermint oil/caraway oil capsules avoids subjective discomfort to the patient caused by gastroesophageal reflux. In order to confirm bioequivalence of an enteric-coated formulation containing peppermint oil and caraway oil and an immediate release formulation of both oils, the pharmacokinetics of menthol and carvone after oral administration of the two formulations were studied in a randomized, two-period crossover study in 16 healthy male volunteers. The subjects received 180 mg peppermint oil and 100 mg caraway oil, once as 2 enteric coated capsules of the fixed combination preparation containing 90 mg peppermint oil and 50 mg caraway oil each (test) and once in the form of 5 capsules of an immediate release formulation (reference) containing 36 mg peppermint oil and 20 mg caraway oil each.

The capsules were taken with 250 ml water after a 10 hour fast. Both substances were determined in plasma by GC/MS after extraction. The limit of quantification was 10 ng/ml for menthol and 0.5 ng/ml for carvone. The mean maximum plasma levels for menthol were 1196 ng/ml after administration of the test medication and 1492 ng/ml after administration of the reference medication. The bioavailability with respect to the AUC was comparable after administration of test and reference preparation, the 90% confidence interval was 97 to 105%. As expected, there were considerable differences for Tₘₐₓ. After application of the enteric-coated form, the maximum concentration was reached significantly later (3.0 hour vs. 1.7 hour) compared to the immediate release capsule. Corresponding data also were calculated for carvone. After application of the test medication, the maxima of 14 ng/ml for both formulations were reached later (2.5 hour vs. 1.3 hour). The 90% confidence interval of the AUC for carvone was 79 to 119% and therefore in the acceptable range for bioequivalence at 80 to 125%.

See the following related figures:

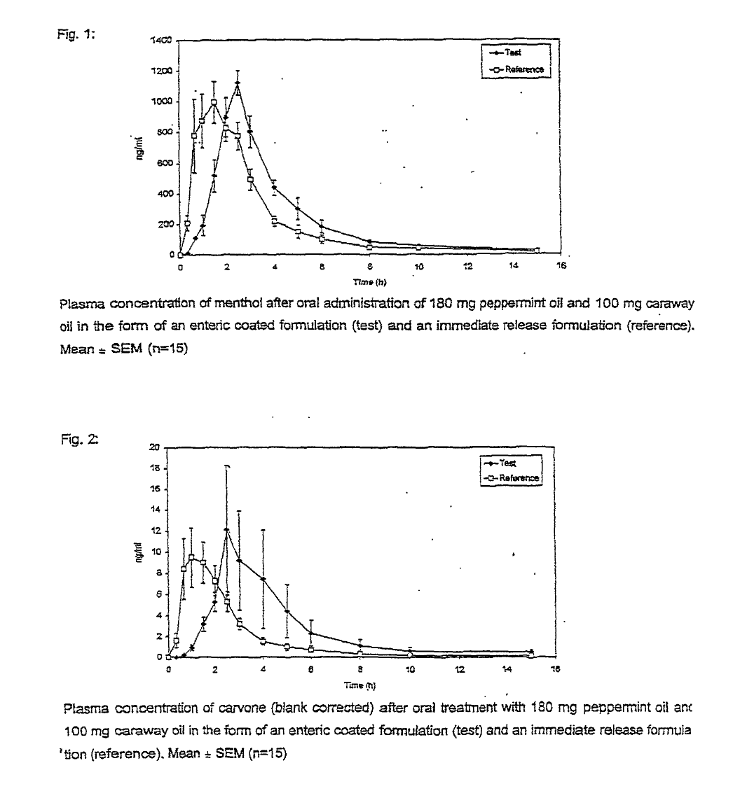

FIG. 1—Plasma concentration of menthol after oral administration of 180 mg peppermint oil and 100 mg caraway oil in the form of an enteric coated formulation (test) and an immediate release formulation (reference). Mean±SEM (n=15).

FIG. 2—Plasma concentration of carvone (blank corrected) after oral treatment with 180 mg peppermint oil and 100 mg caraway oil in the form of an enteric coated formulation (test) and an immediate release formulation (reference). Mean±SEM (n=15).

### EXAMPLE 7

Comparison of the tolerance of highly-dosed caraway oil-peppermint oil capsules with and without a gastric juice-resistant coating. Respective dosages in this Example are 90 mg peppermint oil and 50 mg caraway oil.

a) In an open study with a non-gastric juice-resistant capsule containing peppermint oil and caraway oil which included 200 patients, 17 patients (i.e, 8.5%) reported undesired effects such as pyrosis, belches leaving a peppermint taste behind, and gastric complaints.

b) In a double blind study with gastric-juice resistant peppermint oil-caraway oil capsules in 80 patients, only 3% reported the above-mentioned side-effects.

The foregoing description of the present invention is merely illustrative thereof, and it is understood that variations and modification can be made without departing from the spirit or scope of the invention.

We claim:

1. A pharmaceutical preparation for treating dyspeptic complaints and spasmodic complaints in the gastrointestinal region, comprising therapeutic amounts of peppermint oil and caraway oil in a delayed release form.

2. A pharmaceutical preparation of claim 1 wherein the peppermint oil is present in an amount of about 50 mg to 180 mg by weight.

3. A pharmaceutical preparation of claim 1 wherein the peppermint oil is present in an amount of about 80 mg to 150 mg by weight.

4. A pharmaceutical preparation of claim 1 wherein the peppermint oil is present in an amount of about 90 mg by weight.

5. A pharmaceutical preparation of claim 1 wherein the caraway oil is present in an amount of about 25 mg to 100 mg by weight.

6. A pharmaceutical preparation of claim 1 wherein the caraway oil is present in an amount of about 45 mg to 75 mg by weight.

7. A pharmaceutical preparation of claim 1 wherein the caraway oil is present in an amount of about 50 mg by weight.

8. A pharmaceutical preparation of claim 1 wherein peppermint oil is present in amount of about 90 mg by weight and caraway oil is present in an amount of about 50 mg by weight.

9. A pharmaceutical preparation of claim 1 wherein the preparation has substantially the same bioavailability with respect to reabsorbed quantity (AUC) as compared to an immediate release preparation comprising therapeutic amounts of peppermint oil and caraway oil.

10. A pharmaceutical preparation of claim 1 wherein the preparation comprises an enteric-coating.

11. A pharmaceutical preparation of claim 1 wherein the preparation comprises an enteric-coated soft gelatin capsule.

12. A pharmaceutical preparation of claim 10, wherein the enteric coating comprises a cellulosephthalate derivative or hydroxypropylmethylcellulosesuccinate.

13. A pharmaceutical preparation of claim 12, wherein the cellulosephthalate derivative comprises celluloseacetatephthalate.

14. A pharmaceutical preparation of claim 10, wherein the enteric coating comprises a shellac.

15. A pharmaceutical preparation of claim 10, wherein the enteric coating comprises an aqueous-based shellac.

16. A pharmaceutical preparation of claim 10 wherein the enteric coating further comprises one or more of a plasticizer and antisticking agent.

17. A pharmaceutical preparation of claim 1 wherein the preparation affects gastric motility.

18. A pharmaceutical preparation of claim 1 wherein the preparation has substantial bioequivalence as compared to an immediate release capsule comprising therapeutic amounts of peppermint oil and caraway oil.

19. A pharmaceutical preparation for treating dyspeptic complaints and spasmodic complaints in the gastrointestinal region, comprising therapeutic amounts of peppermint oil and caraway oil in a delayed release soft gelatin capsule having an enteric-coating thereon.

20. A pharmaceutical preparation of claim 19 wherein peppermint oil is present in amount of about 90 mg by weight and caraway oil is present in an amount of about 50 mg by weight.

21. The pharmaceutical preparation of claim 19 wherein the enteric coating comprises a shellac.

22. The pharmaceutical preparation of claim 19 wherein the enteric coating comprises an aqueous-based shellac.

23. A method of treating dyspeptic complaints and spasmodic complaints in the gastrointestinal region, comprising administering to a mammal a pharmaceutical preparation comprising therapeutic amounts of peppermint oil and caraway oil in a delayed release form.

24. The method of claim 23 wherein the peppermint oil is present in the preparation in an amount of about 50 mg to 180 mg by weight.

25. The method of claim 23 wherein the peppermint oil is present in the preparation in an amount of about 90 mg by weight.

26. The method of claim 23 wherein the caraway oil is present in the preparation in an amount of about 25 mg to 100 mg by weight.

27. The method of claim 23 wherein the caraway oil is present in the preparation in an amount of about 50 mg by weight.

28. The method of claim 23 wherein the peppermint oil is present in the preparation in amount of about 90 mg by weight and caraway oil is present in an amount of about 50 mg by weight.

29. The method of claim 23 wherein the preparation has substantially the same bioavailability with respect to reabsorbed quantity (AUC) as compared to an immediate release preparation comprising therapeutic amounts of peppermint oil and caraway oil.

30. The method of claim 23 wherein the preparation further comprises an enteric-coating.

31. The method of claim 23 wherein the preparation further comprises an enteric-coated soft gelatin capsule.

32. The method of claim 30 wherein the enteric coating comprises a cellulosephthalate derivative.

33. The method of claim 30 wherein the enteric coating comprises a shellac.

34. The method of claim 30 wherein the enteric coating comprises an aqueous-based shellac.

35. The method of claim 23 wherein the preparation affects gastric motility in the mammal.

36. The method of claim 23 wherein the preparation has substantial bioequivalence in the mammal as compared to an immediate release capsule comprising therapeutic amounts of peppermint oil and caraway oil.
